(19) United States
(12) Patent Application Publication
(10) Pub. No.: US 2024/0245532 A1
(43) Pub. Date: Jul. 25, 2024

(54) PROSTHETIC DIGIT ACTUATOR

(71) Applicant: Touch Bionics Limited, Livingston (GB)

(72) Inventors: Rodrigo Mercader Rivera, Livingston (GB); Hugh Gill, Paisley (GB)

(21) Appl. No.: 18/429,095

(22) Filed: Jan. 31, 2024

Related U.S. Application Data
(63) Continuation of application No. 17/098,045, filed on Nov. 13, 2020, now Pat. No. 11,931,270.
(60) Provisional application No. 62/935,852, filed on Nov. 15, 2019.
(60) Provisional application No. 63/064,614, filed on Aug. 12, 2020.

(51) Int. Cl. A61F 2/58; A61F 2/68

(52) U.S. Cl. CPC A61F 2/586; A61F 2/68; A61F 2002/587; A61F 2002/6836

(57) ABSTRACT

Features for a prosthetic digit actuator. The various systems and methods allow for smaller volume actuators, which in turn allows for smaller digits and/or more space for other features of the digit. The actuator includes a motor that causes rotation of a worm gear along a fixed worm wheel. The worm gear is unibody with the output shaft. The worm gear climbs along the worm wheel to cause rotation of a digit or digit segment. The arrangement of the actuator parts allows for transmitting axial forces in first and second directions corresponding respectively to performing opening and closing rotations of the digits.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, the present application is a continuation of U.S. patent application Ser. No. 17/098,045, titled “PROSTHETIC DIGIT ACTUATOR” and filed on Nov. 13, 2020, which claims priority to U.S. Provisional Patent Application No. 62/935,852, titled “PROSTHETIC DIGIT ACTUATOR” and filed on Nov. 15, 2019, and to U.S. Provisional Patent Application No. 63/064,614, titled “PROSTHETIC DIGIT ACTUATOR” and filed on Aug. 12, 2020, each of which is incorporated herein by reference in its entirety for all purposes and forms a part of this specification.

### BACKGROUND

### Field

The disclosure relates to prosthetic digits, in particular to actuators for prosthetic digits.

### Description of the Related Art

Prosthetics are used to replace amputated natural body parts. Prosthetic digits may be used to replace amputated fingers and thumbs on a hand, or with prosthetic hands and/or arms. Existing solutions for prosthetic digits require large amounts of power and volume. Improvements to these and other drawbacks are desirable.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for prosthetic digit actuators.

The following disclosure describes non-limiting examples of some embodiments. Other embodiments of the disclosed systems and methods may or may not include the features described herein. Moreover, disclosed advantages and benefits can apply only to certain embodiments of the invention and should not be used to limit the disclosure.

Features for a prosthetic digit actuator are described. The various systems and methods allow for smaller volume actuators, which in turn allows for smaller digits and/or more space for other features of the digit. Such digits may be useful for smaller amputees having smaller hands, and for children with amputated digits and/or hands. The actuator includes a motor that causes rotation of a worm gear along a fixed worm wheel. A gearbox may be transmit the rotation. The worm gear is axially fixed along the output shaft. The worm gear climbs along the worm wheel to cause rotation of the digit and/or digit segment. A thrust bearing is located an on outer side of the worm gear relative to the motor along the shaft. A radial bearing is located between the worm gear and thrust bearing. The arrangement of the actuator parts allows for transmitting axial forces in first and second directions corresponding respectively to performing opening and closing rotations of the digits.

In one aspect, an actuator for a prosthetic digit is described. The actuator comprises a housing, a motor, an output shaft, a worm gear, a radial bearing, a thrust bearing, and a worm wheel. The motor is supported within the housing. The output shaft extends proximally along a rotation axis, where the motor is in mechanical communication with the output shaft and is configured to cause a rotation of the output shaft about the rotation axis. The worm gear is supported along the output shaft, and the worm gear is axially unsupported on a distal-facing side of a distal end of the worm gear, with the output shaft configured to cause rotation of the worm gear about the rotation axis, and the worm gear axially fixed on the output shaft. The radial bearing is supported along the output shaft proximally of the worm gear, with the radial bearing comprising an inner race in mechanical communication with an outer race, and the outer race in mechanical communication with the housing and rotationally fixed relative to the housing. The thrust bearing is supported along the output shaft proximally of the radial bearing, with the thrust bearing comprising a proximal race in mechanical communication with a distal race, the distal race in mechanical communication with and rotationally fixed relative to the outer race of the radial bearing, and the proximal race supported at a proximal end of the output shaft axially constraining the distal race and configured to rotate relative to the distal race, such that rotation of the output shaft rotates the distal race about the rotation axis. The worm wheel is configured to be attached with a prosthetic hand, where the worm wheel is in mechanical communication with the worm gear such that rotation of the worm gear about the rotation axis causes the worm gear to travel along an arcuate outer perimeter of the worm wheel.

Various embodiments of the various aspects are described. The actuator may further comprise a gearbox, where the motor is configured to rotate the output shaft via the gearbox. The inner race of the radial bearing may be in mechanical communication with the output shaft, and the output shaft may be configured to rotate the inner race relative to the outer race. The proximal end of the worm gear may be configured to transmit axial forces, due to actuation of the actuator, to a distal end of the inner race, which may transmit the axial forces via the outer race to the housing. The proximal race of the thrust bearing may be configured to transmit axial forces, due to actuation of the actuator, to the distal race of the thrust bearing, which may transmit the axial forces via the outer race of the radial bearing to the housing.

In another aspect, as actuator for a prosthetic digit is described. The actuator comprises a motor, an output shaft, a worm gear, a thrust bearing, and a worm wheel. The output shaft is located proximally of the motor, where the motor is configured to cause a rotation of the output shaft. The worm gear is supported along the output shaft, with the output shaft configured to cause rotation of the worm gear about the rotation axis. The thrust bearing is supported along the output shaft proximally of the worm gear. The worm wheel is in mechanical communication with the worm gear, where rotation of the worm gear about the rotation axis causes the worm gear to travel along an arcuate outer perimeter of the worm wheel.

Various embodiments of the various aspects are described. The worm gear may be axially unsupported on a distal-facing side of a distal end of the worm gear. The actuator may further comprise a gearbox in mechanical communication with the motor, where a space is located in between the gearbox and the distal end of the worm gear. The worm gear may be configured to remain axially fixed along the output shaft as the worm gear rotates. The worm gear may be bonded the output shaft. The actuator may further comprise a radial bearing supported along the output shaft in between the worm gear and the thrust bearing. A distal end of the radial bearing may contact a proximal end of the worm gear. The radial bearing may comprise an inner race and an outer race, with the inner race rotatable relative to the outer race, and where the inner race contacts the proximal end of the worm gear, and the outer race is rotationally stationary relative to the housing. The thrust bearing may comprise a distal race and a proximal race, with the distal race in mechanical communication with the radial bearing, and the proximal race configured to rotate relative to the distal race. The actuator may further comprise a cap supported along the output shaft proximally of the thrust bearing. The thrust bearing may comprise a distal race and a proximal race, and the cap may axially constrain the proximal race such that the cap and proximal race are configured to rotate together relative to the distal race. The actuator may further comprise a radial bearing supported along the output shaft in between the worm gear and the thrust bearing.

In another aspect, a prosthetic digit is described. The prosthetic digit comprises, a distal segment, a proximal segment. The proximal segment is rotatably attached to the distal segment and configured to rotatably attach to a prosthetic hand, with the proximal segment comprising a housing and an actuator. The actuator comprises a motor, a worm gear, a thrust bearing, and a worm wheel. The motor is configured to cause rotation of a proximally-extending output shaft. The worm gear is supported along the output shaft, with the output shaft configured to cause rotation of the worm gear about the rotation axis. The thrust bearing is supported along the output shaft proximally of the worm gear. Rotation of the worm gear causes the worm gear to travel along an arcuate outer perimeter of the worm wheel to rotate the proximal segment relative to the prosthetic hand.

Various embodiments of the various aspects, such as the prosthetic digit and other aspects, are described. The worm gear may be axially fixed relative to the output shaft. The worm gear may be bonded to the output shaft. The worm gear and the output shaft may be welded together. The worm gear and the output shaft may be unibody. The worm gear may be axially unsupported on a distally-facing side of a distal end of the worm gear. The prosthetic digit may further comprise a space located between the distally-facing side of the distal end of the worm gear and a proximal end of the motor. The prosthetic digit may further comprise a radial bearing located between the worm gear and the thrust bearing. The thrust bearing may comprise a distal race and a proximal race, with the proximal race configured to rotate with the output shaft relative to the distal race. The prosthetic digit may further comprise a gearbox, wherein the motor is configured to cause rotation of the output shaft via the gearbox. The worm gear may be axially unsupported on a distally-facing side of a distal end of the worm gear. The prosthetic digit further comprise a space located between the distally-facing side of the distal end of the worm gear and proximal end of the gearbox.

In another aspect, a prosthetic digit is described that comprises any of the actuators described herein.

In another aspect, a prosthetic hand is described that comprises any of the prosthetic digits described herein.

In another aspect, an actuator for a prosthetic digit is described. The actuator comprises a housing, a motor, an output shaft, a worm gear, a radial bearing, a thrust bearing, and a worm wheel. The housing is configured to be rotated relative to a prosthetic hand about a knuckle axis. The motor is supported within the housing. The output shaft is in mechanical communication with the motor, where the output shaft extends proximally along a rotation axis and has an outer thread located at a proximal end of the output shaft, where the motor is configured to cause a rotation of the output shaft about the rotation axis. The worm gear is axially fixedly supported along the output shaft and extending from a proximal end to a distal end with an outer threaded portion therebetween, with the distal end of the worm gear spaced axially from the proximal end of the motor to define a space adjacent to the distal end of the worm gear, with the output shaft configured to cause rotation of the worm gear about the rotation axis, and the worm gear configured to remain axially stationary along the output shaft as the worm gear rotates. The radial bearing is supported along the output shaft proximally of the worm gear, with the radial bearing comprising an inner race in mechanical communication with an outer race, the inner race in mechanical communication with the output shaft and configured to rotate relative to the outer race, and the outer race in mechanical communication with the housing and rotationally fixed relative to the housing. The output shaft is configured to rotate the inner race relative to the outer race, where the proximal end of the worm gear is configured to transmit axial forces due to actuation of the actuator to a distal end of the inner race which transmits the axial forces via the outer race to the housing. The thrust bearing is supported along the output shaft proximally of the radial bearing, with the thrust bearing comprising a proximal race in mechanical communication with a distal race. The distal race is in mechanical communication with and rotationally fixed relative to the outer race of the radial bearing, and the proximal race is configured to rotate relative to the distal race and is supported along the output shaft at the proximal end of the output shaft. The proximal race has an inner thread engaging the outer thread of the output shaft to axially constrain the distal race, such that rotation of the output shaft rotates the proximal race of the thrust bearing about the rotation axis. The worm wheel has outer teeth extending along an arcuate outer perimeter of the worm wheel and in mechanical communication with the outer threaded portion of the worm gear. The worm wheel is configured to be fixedly attached with a prosthetic hand, where rotation of the worm gear about the rotation axis causes the worm gear to travel along the arcuate outer perimeter of the worm wheel such that the housing, the motor, the output shaft and the worm gear rotate about the knuckle axis.

Various embodiments of the various aspects are described. The knuckle axis may be parallel to the rotation axis of the output shaft. The actuator may further comprise a gearbox, where the motor is configured to rotate the output shaft via the gearbox. The distal end of the worm gear may be spaced axially from a proximal end of the gearbox to define the space adjacent to the distal end of the worm gear.

In another aspect, an actuator for a prosthetic digit is described. The actuator comprises a housing, a motor, an output shaft, a radial bearing, a 4-point contact bearing, and a worm wheel. The motor is supported within the housing. The output shaft extends proximally along a rotation axis. The motor is in mechanical communication with the output shaft. The motor is configured to cause a rotation of the output shaft about the rotation axis. The output shaft includes a unibody worm gear axially fixed on the output shaft. The radial bearing is supported along the output shaft proximally of the worm gear. The radial bearing comprises an inner race in mechanical communication with an outer race. The outer race is in mechanical communication with the housing. The outer race is rotationally fixed relative to the housing. The 4-point contact bearing is located at a distal end of the output shaft distally of the worm gear. The 4-point contact bearing comprises at least one outer race and at least one inner race. The at least one outer race contacts a step on an inner sidewall of the housing that prevents distal translation of the 4-point contact bearing. The at least one inner race contacts the distal end of the output shaft. The at least one inner race is configured to rotate relative to the at least one outer race, such that rotation of the output shaft rotates the at least one inner race about the rotation axis. The worm wheel is configured to be attached with a prosthetic hand. The worm wheel is in mechanical communication with the worm gear such that rotation of the worm gear about the rotation axis causes the worm gear to travel along an arcuate outer perimeter of the worm wheel to thereby rotate the housing about the worm wheel.

Various embodiments of the various aspects are described. The actuator may further comprise a carrier shaft. The carrier shaft may extend proximally. The carrier shaft may be configured to engage the output shaft to mechanically transmit rotation from the motor to the output shaft. The output shaft may comprise an internal opening extending axially at least partially therethrough. The output shaft may be configured to at least partially receive the carrier shaft therein. The internal opening may comprise internal threads. The carrier shaft may comprise external threads configured to engage the internal threads. The actuator may further comprise a gearbox. The motor may be configured to rotate the output shaft via the gearbox. The at least one inner race may comprise two inner races. The at least one outer race may comprise two outer races. The two inner races may contact and rotate with the output shaft. The two outer races may be axially compressed by the housing and a preload ring.

In another aspect, an actuator for a prosthetic digit is described. The actuator comprises a housing, a motor, an output shaft, a first bearing, a second bearing, and a worm wheel. The motor is supported within the housing. The output shaft has a unibody worm gear. The output shaft extends proximally along a rotation axis. The motor is in mechanical communication with the output shaft. The motor is configured to cause a rotation of the output shaft about the rotation axis. The first bearing is located at a proximal end of the output shaft proximally of the worm gear. The second bearing is located at a distal end of the output shaft distally of the worm gear. The worm wheel is configured to be attached with a prosthetic hand. The worm wheel is in mechanical communication with the worm gear such that rotation of the worm gear about the rotation axis causes the worm gear to travel along the worm wheel to cause the housing and motor to rotate about the worm wheel.

Various embodiments of the various aspects are described. The actuator may further comprise a preload ring. The preload ring may be configured to axially constrain the second bearing. The housing may further comprise an inward step on a inner surface. The inward step may prevent axial movement of the second bearing in the distal direction. The first bearing may be a radial bearing. The first bearing may comprise an inner race in mechanical communication with an outer race. The outer race may be in mechanical communication with the housing. The outer race may be rotationally fixed relative to the housing. The second bearing may be a 4-point contact bearing. The second bearing may comprise at least one outer race and at least one inner race. The at least one outer race may contact a step on an inner sidewall of the housing that prevents distal translation of the 4-point contact bearing. The at least one inner race may contact the distal end of the output shaft. The at least one inner race may be configured to rotate relative to the at least one outer race, such that rotation of the output shaft rotates the at least one inner race about the rotation axis. Rotation of the worm gear about the rotation axis may cause the worm gear to travel along an arcuate outer perimeter of the worm wheel. The worm gear may be axially unsupported on a distal-facing side of a distal end of the worm gear. The worm gear may be axially unsupported on a proximal-facing side of a proximal end of the worm gear. The actuator may further comprise a gearbox in mechanical communication with the motor, where a space is located in between the gearbox and the distal end of the worm gear.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawing, can be arranged, substituted, combined, and designed in a wide variety of different configurations, all of which are explicitly contemplated and make part of this disclosure.

FIG. 1 is a front view of a lower arm stump having embodiments of prosthetic digits attached thereto, which prosthetic digits may be any of the prosthetic digits including any of the actuators described herein.

FIGS. 2A-2B are back and front views, respectively, of a prosthetic hand incorporating embodiments of prosthetic digits, which prosthetic digits may be any of the prosthetic digits including any of the actuators described herein.

FIGS. 3A-3B are rear and front perspective views respectively of the prosthetic digit of FIGS. 1-2B having an actuator therein.

FIGS. 4A-4C are exploded views of the actuator of the prosthetic digit of FIGS. 1-3B.

FIG. 5 is a side cross-section view of the prosthetic digit of FIGS. 1-3B,

FIG. 6 is a perspective view of a gear box and output shaft of the actuator of FIGS. 4A-5.

FIGS. 7A-7D are various views of a worm gear of the actuator of FIGS. 4A-5.

FIGS. 8A-8B are side and cross-section views, respectively, of an embodiment of a space between a worm gear and gearbox that may be implemented with the various actuators described herein.

FIGS. 9A-9B are side and cross-section views, respectively, of another embodiment of a space between a worm gear and gearbox that may be implemented with the various actuators described herein.

FIGS. 10A-10G are various views of another embodiment of a prosthetic digit having an actuator where the output shaft has a unibody worm gear.

FIG. 11 is a partial exploded view of the actuator of the prosthetic digit of FIGS. 10A-10G.

FIG. 12 is a cross-section view of the output shaft of the actuator from FIGS. 10A-11.

### DETAILED DESCRIPTION

The following detailed description is directed to certain specific embodiments of the development. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments.

Features for prosthetic digit actuators are described. The actuator provides a drive mechanism where a worm wheel is fixed relative to a palm, and a housing via a rotated worm gear rotates around the worm wheel. A motor may rotate over the worm wheel via a gear box and shaft with the worm gear supported along the length of the shaft. The motor may be fixed with the housing, for example bonded or threaded and bonded with the housing. The worm gear is axially fixed, for example unibody, bonded or welded, to the shaft, for instance prior to gearbox assembly. “Unibody” as used herein refers to a monolithic piece, which for example could result from being machined from the same stock piece of raw material. Thus the shaft and worm gear may be a single piece component, for example machined from the same piece of metal.

In some embodiments, the worm gear is located in between the motor and a thrust bearing. The worm gear may be spaced from the motor or gearbox, for example spaced no less than 0.040 mm. The worm gear backs against a radial bearing, to allow for unloading first axial forces in a first direction away from the motor. These first axial forces may be due to rotation of the digit in a first direction, such as a closing rotation of the digit. The radial bearing transmits these first axial forces to the housing because an outer race of the radial bearing is fixed, e.g. bonded, to the housing. An end of the shaft is threaded and is thereby fixed to a proximal race of the thrust bearing, which may be a cap or nut. The proximal race unloads second axial forces in a second direction toward the motor which are opposite respectively to the first axial forces and direction. The second axial forces are unloaded to a distal race of the thrust bearing, which in turn unloads on the non-rotating element of the radial bearing, which unloads on the housing. The actuator may axially constrain the shaft so that no axial forces are transmitted to the motor/gearbox and no axial play is present. Axial play is eliminated by threading the proximal thrust bearing race to the shaft, for example during assembly.

The actuator has various uniquely desirable attributes. For example, the use of a thrust bearing reduces friction losses under axial load in the direction away from the motor, thus allowing faster digit closure compared to a plain bearing. As further example, the use of a thrust bearing located on the end of the shaft to deal with forces in the direction away from the motor is contrary to typical design practice. This is in contrast, for example, to a thrust bearing being located between the motor and the worm gear. The unique configuration described herein includes the worm gear being fixed to the shaft and the shaft being constrained axially. As further example, the configuration described herein minimizes the length of the digit. The configuration thus saves space and allows for a shorter and smaller digit, for example by not needing to accommodate the length of the thrust bearing in between the worm gear and the motor, and by having the shaft with the worm gear and bearings thereon extending toward the hand. These are just some example attributes, and others are described herein.

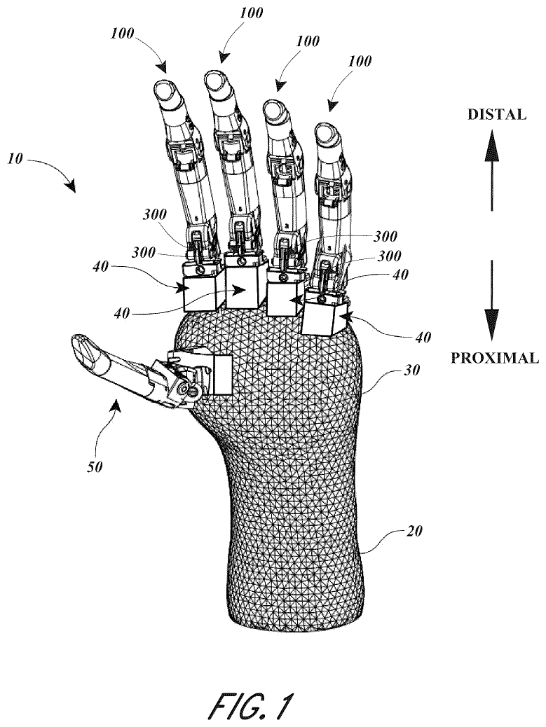

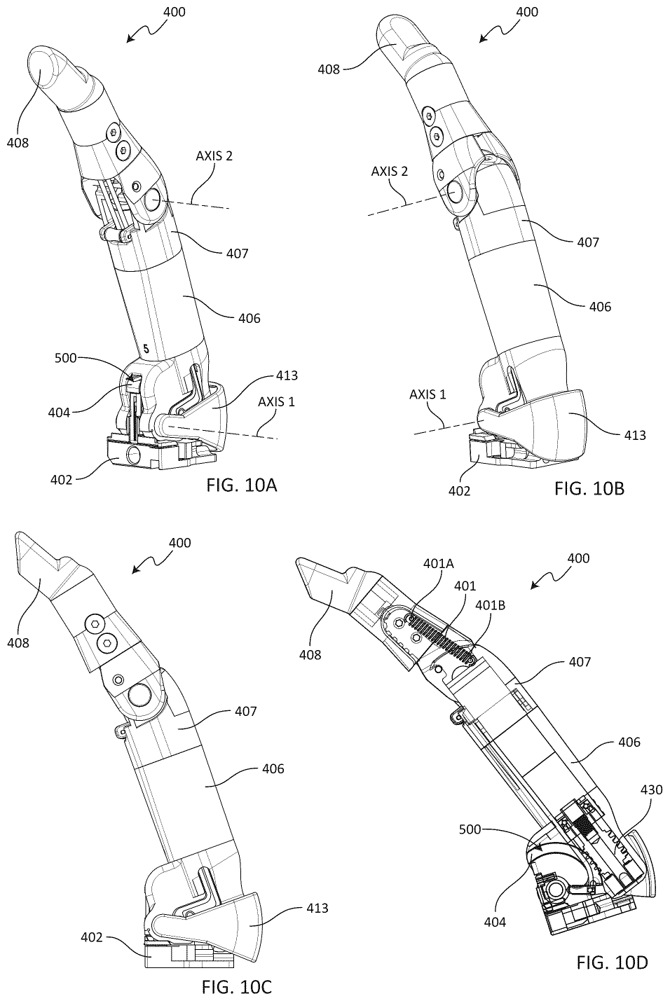

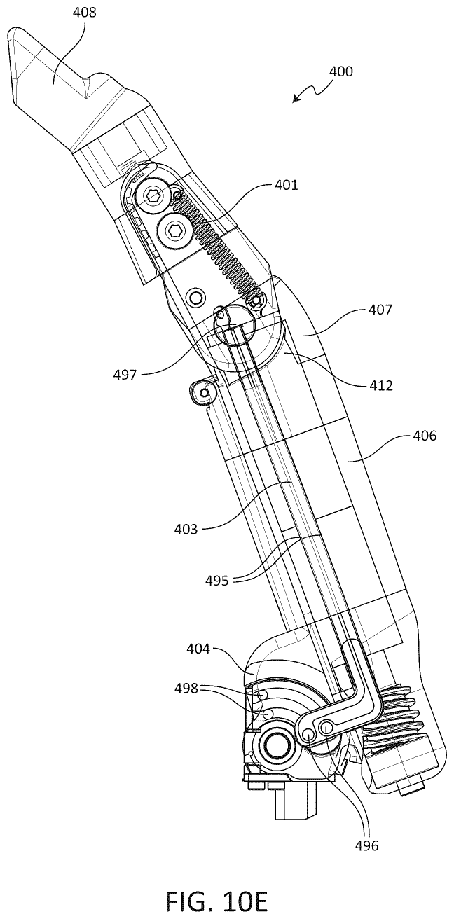

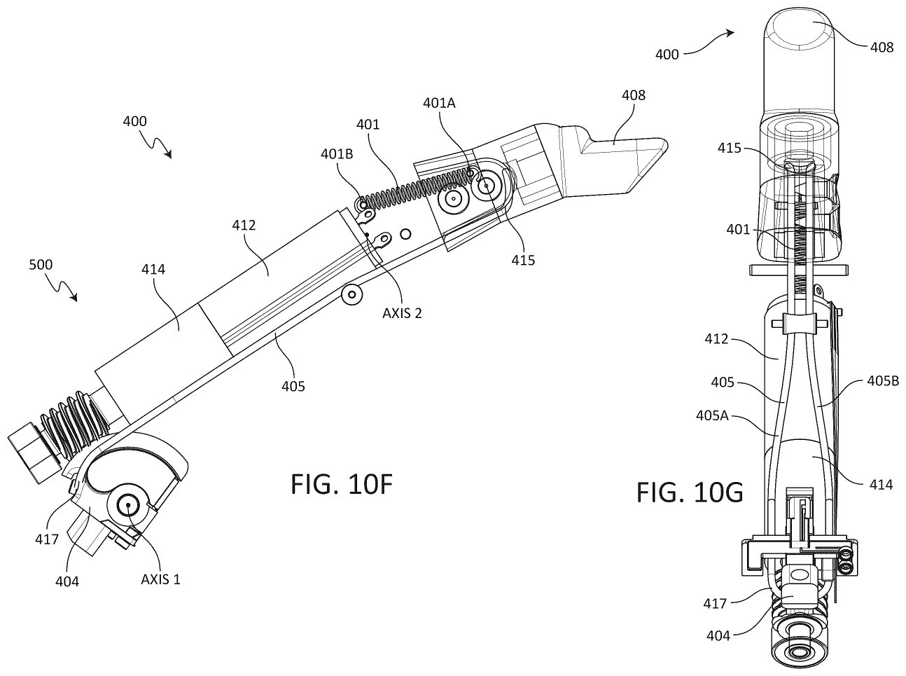

In some embodiments, as shown in FIGS. 10A-12, the actuator includes a distal bearing on a distal end of the output shaft and supporting the output shaft within the housing. The bearing may support the shaft radially, axially, or radially and axially. The bearing may be a 4-point contact bearing that provides both axial and radial support. A preload ring may secure the bearing within the housing. The worm gear may be unibody with the output shaft to form a “unibody shaft” and be located proximally of the distal bearing when the distal bearing is assembled onto the shaft. A proximal bearing may be located on a proximal end of the output shaft and support the output shaft. The proximal bearing may be a radial bearing. A proximal end of the output shaft may be internally threaded to mate with an external thread of a carrier shaft of the gear box. The embodiments of the prosthetic digit and actuator of FIGS. 10A-12 may include any of the features and/or functions as described with respect to the embodiments of the prosthetic digit and actuator of FIGS. 1-9B, and vice versa, except as otherwise stated explicitly or by context.

FIG. 1 is a front or palm-side view of a lower arm prosthetic system 10 including a lower arm stump 20, having four prosthetic digits 100 with an actuator 300 therein, and a prosthetic thumb 50, attached to the stump 20. The prosthetic digits 100 may be any of the prosthetic digits described herein and include any of the actuators described herein. In some embodiments, the thumb 50 may be any of the prosthetic digits described herein and include any of the actuators described herein. There may be one, two, three, four or more of the digits 100, with each digit 100 having any of the actuators described herein. The digits 100 may be connected to a residual natural palm 30, as shown in FIG. 1. In some embodiments, the digits 100 may be connected to the end of a lower arm stump 20, or to a prosthetic hand, or to a partial prosthetic hand. The digits 100 may include the actuator features described herein to provide small digits that take up smaller volumes compared to digits with other actuator features, among other advantages.

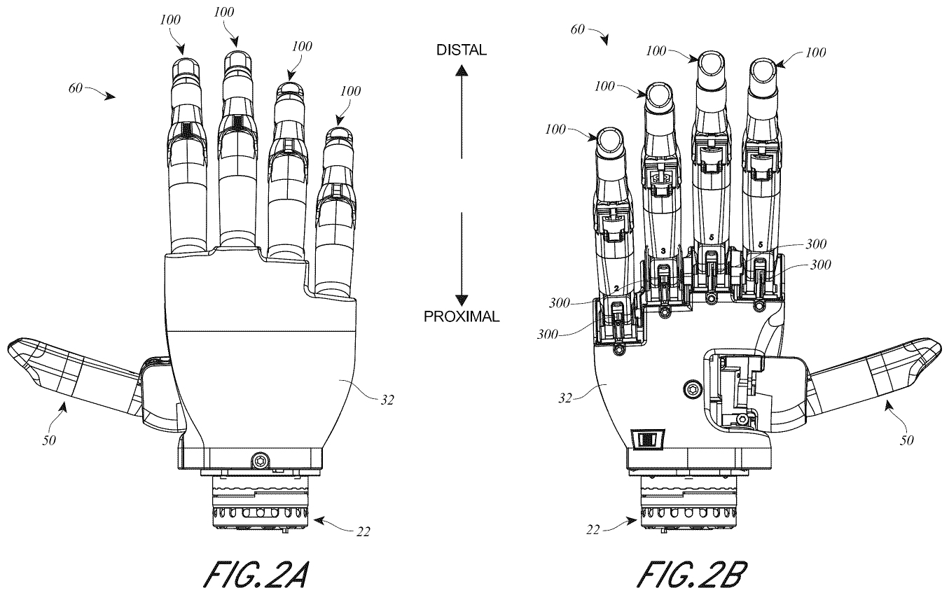

FIGS. 2A-2B are back and front views, respectively, of a prosthetic hand 60 incorporating the prosthetic digits 100 and the prosthetic thumb 50. The hand 60 has a palm portion 32 attached to proximal ends of the digits 100 and thumb 50. The hand 60 may have a wrist 22 that may rotate, which may allow for rotation of the palm portion 32, and the digits 100 and thumb 50 attached thereto, about a longitudinal axis defined by the wrist 22. The prosthetic digits 100 may be any of the prosthetic digits described herein and include any of the actuators described herein to cause rotation of the digits, such as opening and closing rotations of the digits or digit segments.

In some embodiments, the lower arm prosthetic system 10, the digits 100, the hand 60, the wrist 22, and the thumb 50 may include any of the features, respectively, for a lower arm prosthetic system, digits, hand, wrist, or thumb, described for example, in U.S. Provisional Patent Application No. 62/832,166, filed Apr. 10, 2019, and titled PROSTHETIC DIGIT WITH ARTICULATING LINKS, in U.S. Provisional Patent Application No. 62/850,675, filed May 21, 2019, and titled ACTUATION SYSTEMS FOR PROSTHETIC DIGITS, in U.S. Provisional Patent Application No. 62/902,227, filed Sep. 18, 2019, and titled PROSTHETIC DIGIT ACTUATORS WITH GEAR SHIFTING, in U.S. Provisional Patent Application No. 62/782,830, filed Dec. 20, 2018, and titled ENERGY CONSERVATION OF A MOTOR-DRIVEN DIGIT, in U.S. patent application Ser. No. 16/011,108, filed Jun. 18, 2018, and titled PROSTHETIC DIGIT FOR USE WITH TOUCHSCREEN DEVICES, in U.S. patent application Ser. No. 14/765,638, filed Aug. 4, 2015, and titled MULTI-MODAL UPPER LIMB PROSTHETIC DEVICE CONTROL USING MYOELECTRIC SIGNALS, in U.S. patent application Ser. No. 16/423,802, filed May 28, 2019, and titled WRIST DEVICE FOR A PROSTHETIC LIMB, in U.S. patent application Ser. No. 16/204,059, filed Nov. 29, 2018, and titled SYSTEMS AND METHODS FOR PROSTHETIC WRIST ROTATION, in U.S. Provisional Patent Application No. 62/599,559, filed Dec. 15, 2017, and titled POWERED PROSTHETIC THUMB, in U.S. patent application Ser. No. 16/249,696, filed Jan. 16, 2019, and titled SYSTEMS AND METHODS FOR CONTROLLING A PROSTHETIC HAND, the entirety of each of which is incorporated by reference herein for all purposes and forms part of this specification.

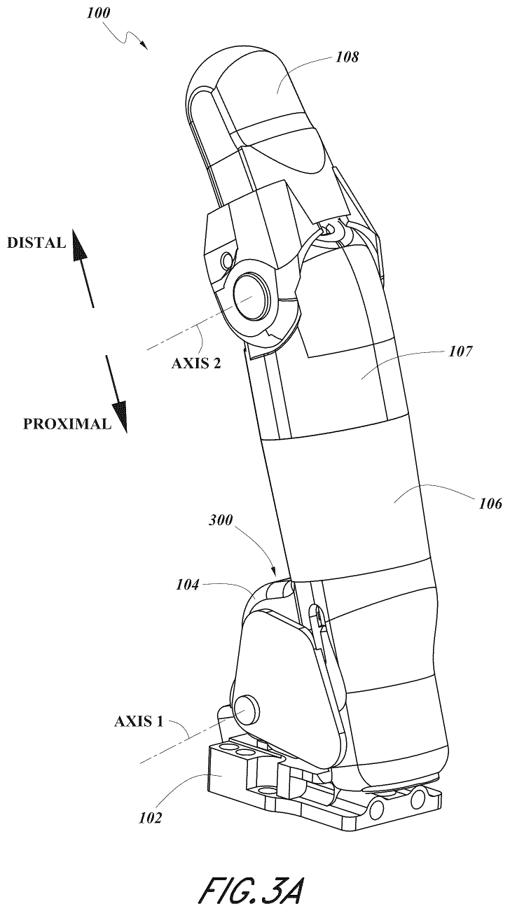

FIGS. 3A-3B are rear and front perspective views, respectively, of the prosthetic digit 100 having the actuator 300. The actuator 300 includes a worm wheel 104, which is partially visible, and is further described herein, for example with respect to FIGS. 4A-7D. As shown in FIGS. 3A-3B, the digit 100 may be described using “proximal” and “distal” directions. Proximal refers to a direction generally toward the hand and away from the tip of the digit 100. Distal refers to a direction generally toward the tip of the digit 100 and away from the hand. For reference, a hand may attach at a base 102 of the digit 100, and at the tip of the digit 100 there may be a distal segment 108. The digit 100 may be used as a small digit compared to typical sized prosthetic digits. The digit 100 may have an overall length as measured in a straight line from a proximal end of the base 102 or from the Axis 1, to a distal tip of the distal end of the digit 100, such as at the distal end of the distal segment 108, with the digit segments fully straightened. This length may be from about 30-90 mm long, from about 40-80 mm long, from about 50-70 mm long, or from about 55-65 mm long.

The digit 100 includes the base 102 at a proximal end thereof. The base 102 is configured to attach to a hand, such as a prosthetic, partial-prosthetic, or natural hand. The digit 100 includes a housing 106, which may be a proximal segment of the digit 100, rotatably attached to the base 102 about a first axis 1. Actuation of the actuator 300 causes the housing 106 to rotate about the first axis 1. The worm wheel 104 remains stationary as the housing 106 rotates about the worm wheel 104. The digit 100, for example the housing 106, may include a distal portion 107 at a distal end of the housing 106. The digit 100 includes a distal segment 108 rotatably attached to the distal portion 107 about a second axis 2. A distal end of the distal portion 107 is attached to a proximal end of the distal segment 108. The segments 106, 108 may rotate relative to each other about the second axis 2. In some embodiments, there may be one, three or more rotatable segments of the digit 100, with one, three or more rotation axes per digit 100. The distal portion 107 may be a separate component of the housing 106 that is attached together or these may be a single structure. The housing 106 may be relatively small compared to typical digits, for example for use with a small or a extra small digit 100. The housing 106 may be from about 10-20 mm in width, from about 12-18 mm in width, or from about 14-16 mm in width. The width may be measured perpendicular to a longitudinal axis of the housing 106, said axis shown for example in FIGS. 4B and 4C. The housing 106 may be from about 30-60 mm long, from about 35-55 mm long, or from about 40-50 mm long. The length may be measured along the longitudinal axis from a proximal end to a distal end of the housing 106.

FIGS. 4A-4C are exploded views of an embodiment of the actuator 300.

FIG. 4A shows a fully exploded view of the actuator 300, and FIGS. 4B and 4C show partial exploded views of the actuator 300. The actuator 300 includes the housing 106 defining an opening 109 therein. The opening 109 may receive the various components of the actuator 300 therein, and provide structural cover. The housing 106 includes a clevis 110 with two projections extending outward therefrom, for example perpendicular to a longitudinal axis defined by the opening 109, and defining a space 111 between the two projections. The two projections of the clevis 110 each include an opening 80 extending therethrough, which can receive a bushing or axle to provide rotation about the first axis 1. The opening 109 may have various inner widths to accommodate the various parts therein. The opening 109 may have a maximum inner width, e.g. a maximum inner diameter, from about 6-14 mm, from about 8-12 mm, or from about 9-11 mm.

The actuator 100 includes the worm wheel 104, which may be partially shaped as a lug or other projection. The worm wheel 104 attaches at a proximal end to the base 102. The worm wheel 104 has an upper portion as oriented in the figure that extends arcuately with a series of teeth 105 thereon. The teeth 105 provide a structure over which a worm gear 130 can engage and travel or climb to effectuate rotation about the first axis 1. The worm wheel 104 may be received into the space 111 defined by the clevis 110 when assembled. The teeth 105 may extend along a circular or other rounded path. The teeth 105 may extend for about ninety degrees about the first axis 1, or other angular amounts. The first axis 1 may be in other locations. The first axis 1 may be fixed, for example where the teeth 105 extend along a circular path. In some embodiments, the first axis 1 may move, for example where the teeth 105 extend along a non-circular, such as an oval or elliptical, path.

The actuator 300 includes a motor 112 and a gearbox 114. The motor 112 may be an electric motor electrically connected to a power source, such as batteries. The motor 112 may be a brushed, brushless, and/or a direct current motor, such as those manufactured by Maxon Motor AG (Switzerland). The gearbox 114 may be a variety of different suitable gearboxes. The motor 112 attaches to the gearbox 114 to provide rotation of a shaft 120, such as an output shaft. The shaft 120 is an elongated structure extending proximally from the motor. The shaft 120 may be rotated at constant or varying torque and/or speed. In some embodiments, there may just be the motor 112 without the gearbox 114. The gearbox 114 may transmit rotation from the motor 112 to the shaft 120, for example to provide a desired torque and/or speed of rotation of the shaft 120. A distal end of the shaft 120 attaches to a proximal end of the gearbox 114. The shaft 120 includes a head 122 at a distal end thereof that forms a disc-like flange. A smaller-diameter shaft portion 124 extends proximally from the head 122 and toward the palm when assembled with a hand. The shaft portion 124 is an elongated structure extending proximally from a proximal end of the motor. The shaft portion 124 may extend from about 16-18 mm, or about 17.6 mm, from a proximal end of the motor 112 to a proximal end tip of the shaft portion 124. The shaft portion 124 may extend 10 mm or less, 15 mm or less, 17 mm or less, 19 mm or less, 21 mm or less, or 25 mm or less, from a proximal end of the motor 112 to a proximal end tip of the shaft portion 124. The shaft portion 124 may be about 3 mm in width, e.g. diameter. The shaft portion may be from about 1.5 to about 4.5 mm, from about 2 mm to about 4 mm, or from about 2.5 mm to about 3.5 mm in width. These widths may refer to a diameter of a portion of the shaft portion 124 having a circular cross-section, and/or to a width of a portion thereof having a non-circular cross-section.

The elongated shaft portion 124 includes a first attachment area 126, along which the worm gear 130 may be supported. The worm gear 130 may be fixedly attached to the shaft portion 124 at the first attachment area 126, as further described herein. The first attachment area 126 may be a location along the shaft portion 124 at which the worm gear 130 and/or other components are positioned. The first attachment area 126 may be located closer to the proximal end of the shaft portion 124 than to the head 122, in the middle of the length of the shaft portion 124, or closer to the head 122 than to the proximal end. The first attachment area 126 may have similar or different surface features as the shaft portion 124 adjacent the head 122. The first attachment area 126 may include threads, projections, modified surface roughness, other suitable features, or combinations thereof. The shaft portion 124 adjacent the head 122 may have a circular or other rounded cross-sectional shape. The first attachment area 126 may have a non-circular cross-sectional shape. For example, as further described herein for instance with respect to FIG. 6, the first attachment area 126 may have a “D” cross-sectional shape, with a flat edge and a rounded edge. In some embodiments, the first attachment area 126 may have a circular or other rounded cross-sectional shape.

The shaft portion 124 includes a second attachment area 128 at a proximal end of the shaft 120. The second attachment area 128 may include threads, as shown, and/or other attachment features. The second attachment area 128 may have any of the cross-sectional shapes as described herein with respect to the first attachment area 126. The second attachment area 128 may have the same or similar cross-sectional shape as the first attachment area 126, such as the “D” cross-section. In some embodiments, the second attachment area 128 may have a different cross-sectional shape from the first attachment area 126. A proximal race 160 of a thrust bearing 150 may be attached at the second attachment area 128, as further described herein.

The actuator 300 includes the worm gear 130. The worm gear 130 has a rounded outer cross-sectional shape and extends from a proximal end to a distal end with an outer thread 134 extending around the body. The thread 134 may extend completely or partially between the distal and proximal ends. The thread 134 is configured to mechanically communicate with, for example directly engage, the teeth 105 of the worm wheel 104.

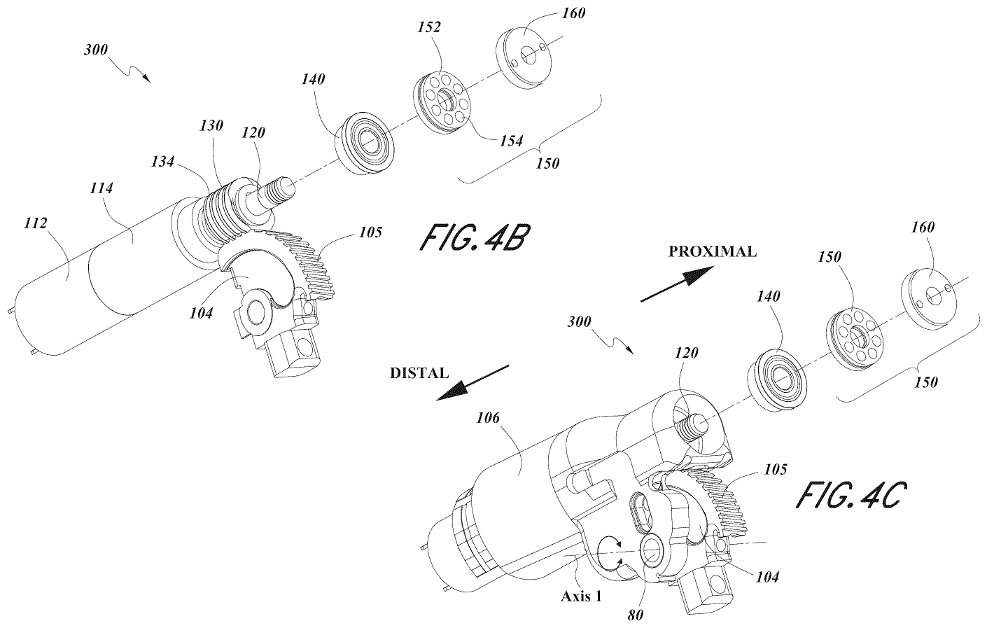

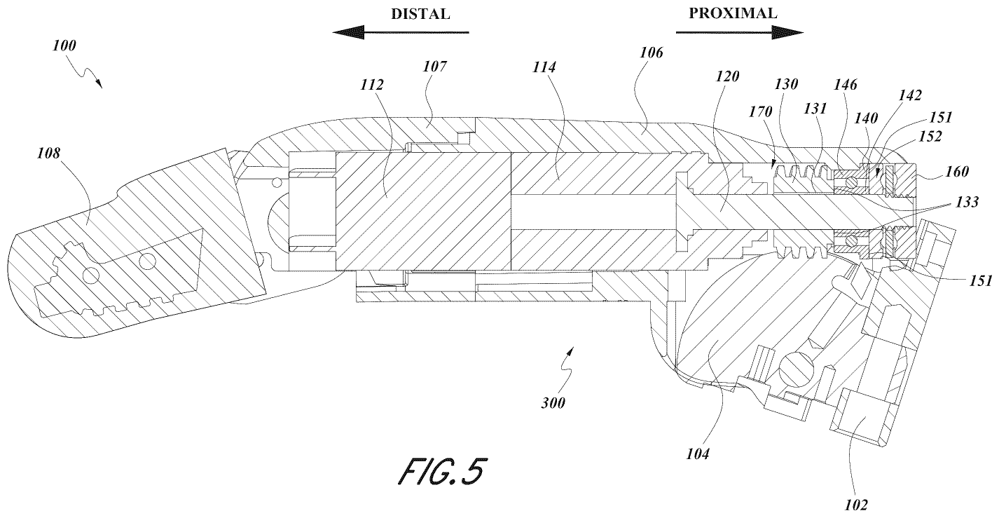

The worm gear 130 defines an axial opening 132 extending therethrough. The opening 132 receives the shaft 120 therein. The opening 132 may have any of the cross-sectional shapes as described herein with respect to the first attachment area 126. The opening 132 may have inner surfaces with an inner cross-sectional shape that corresponds to an outer cross-sectional shape of outer surfaces of the first attachment area 126. The opening 132 may thus have a “D” shaped cross-section to match with a “D” shaped cross-section of the firsts attachment area 126. The cross-sections, such as the “D” cross-section, may correspond in order to transfer rotation of the shaft 120 to the worm gear 130. The “D” cross-section is merely one example and other shapes may be used to transfer such rotation. Further details of the worm gear 130 are described herein, for example with respect to FIGS. 7A-7D.

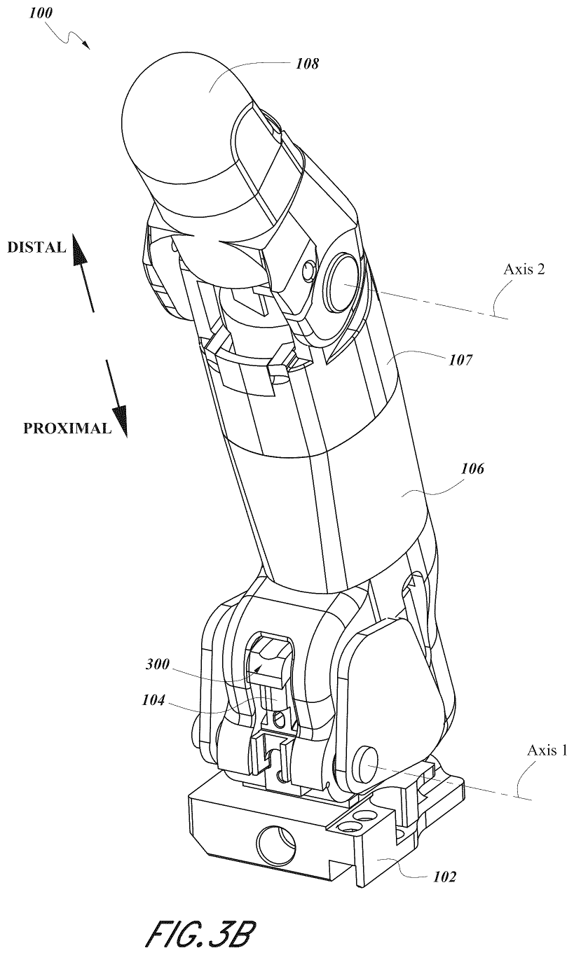

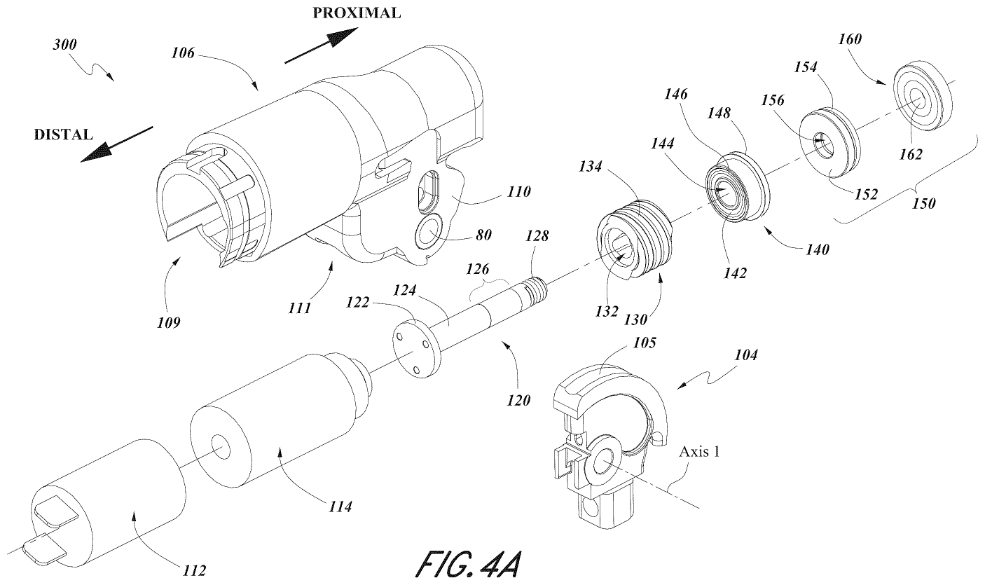

The worm gear 130 is supported by the shaft portion 124, which may be at the first attachment area 126. The worm gear 130 may be bonded to the shaft 120. The worm gear 130 may have an interference fit with the shaft 120. The worm gear 130 may be bonded to the shaft 120, welded with the shaft 120, laser-welded with the shaft 129, interference fitted with the shaft 120, have other suitable mechanical attachment methods with the shaft 120, or combinations thereof, to remain axially fixed on the shaft portion 124. The various mechanical attachment methods may be incorporated at the first attachment area 126. The worm gear 130 may be unibody with the shaft 120, such that the worm gear 130 and the shaft 120 form a single, monolithic part. The worm gear 130 may be fixed at a location along the shaft portion 124 such that a gap is defined between a distal-facing side of the worm gear 130 and an adjacent structure such as the gearbox 114 or the motor 112, as further described herein, for example with respect to FIG. 5.

The actuator 300 includes a radial bearing 140. The radial bearing 140 is located proximally of the worm gear 130. The radial bearing 140 may be a variety of suitable radial bearings configured to transmit radial and/or axial forces from the shaft 120 and/or worm gear 130 to the housing 106. The radial bearing 140 includes an inner race 142 surrounded radially by an outer race 146. The inner and outer races 142, 146 may rotate relative to each other about a longitudinal axis defined by the bearing 140. A series of balls may be located arcuately between the races 142, 146, for example in a radial ball bearing. The inner and outer races 142, 146 may have circular or other rounded inner and outer cross-sectional shapes. The inner race 142 surrounds outer surfaces of the shaft 120 and the outer race 146 is surrounded by inner surfaces of the housing 106. The bearing 140 thus stabilizes the shaft 120 along the length of the shaft 120 and provides for stable rotation of the shaft 120, among other functions. The radial bearing 140 may have an outer diameter, e.g. of the outer race 146, of about 7 mm. This outer diameter may be from about 5 mm to about 9 mm, from about 6 mm to about 8 mm, or from about 6.5 mm to about 7.5 mm.

The inner race 142 defines an opening 144 therethrough. The opening 144 may define the longitudinal axis about which the races 142, 146 rotate relative to each other. The opening 144 is configured to receive a portion of the shaft 120 therein such that the shaft 120 supports the bearing 140 along the length of the shaft portion 124. The opening 144 may thus receive the shaft portion 124 therein. The opening 144 may have a circular cross-sectional shape. The opening 144 may have any of the cross-sectional shapes as described herein with respect to the first attachment area 126, such as “D” shape, etc. The opening 144 may be located at a distal portion of the shaft portion 124. The inner race 142 may be located at or near the first attachment area 126. The inner race 142 may be located proximally of the first attachment area 126. The inner race 142 may be in other locations along the length of the shaft 120.

The inner race 142 may have a transitional fit with the shaft portion 124. For example, the inner race 142 may be fitted with the shaft portion 124 by hand. The fit between the inner race 142 and the shaft 124 may not allow for any free relative movement, such as axial, rotational, and/or radial movement, between the inner race 142 and the shaft 124. The inner race 142 may have a transitional fit with the shaft 120, be bonded to the shaft 120, be attached in other suitable mechanical ways to the shaft 120, or combinations thereof. As further described herein, for example with respect to FIG. 5, a distal end of the inner race 142 contacts a proximal end of the worm gear 130 to transmit axial forces in the proximal direction to the housing 106 via the outer race 146. As further described herein, for example with respect to FIG. 5, as the shaft 120 rotates, the inner race 142 may rotate with the shaft 120 and relative to the outer race 146.

The outer race 146 may include a flange 148 at a proximal end thereof. The flange 148 may protrude radially outwardly from the outer race 146. The flange 148 may have a circular or other rounded cross-sectional shape, or other shapes. The outer race 146 may be partially received into a portion of the housing such that a distal side surface of the flange 148 contacts a proximal-facing surface of the housing 106, as further described herein, for example with respect to FIG. 5. The flange 148 may transmit axial forces in the distal direction to the housing 106. A distal end of the outer race 146 may not contact the worm gear 130. As further described, the outer race 146 may be rotationally stationary with respect to the housing 106.

The actuator 300 includes the thrust bearing 150. The thrust bearing 150 is positioned or located proximally of the worm gear 130 and radial bearing 140. The worm gear 130 is thus located in between the gearbox 114 and the thrust bearing 150. The worm gear 130 may be located in between the motor 112 and the thrust bearing 150. By locating the thrust bearing proximally of the worm gear 130, less volume is needed compared to a digit that locates a thrust bearing between the worm gear 130 and the gearbox 114. The digit 100 may thus have a smaller overall length. The fixed worm wheel 104 in conjunction with the proximal location of the thrust bearing 150, and other features of the actuator 300 described herein, contributes to the smaller volume and the other advantages as further described.

The thrust bearing 150 includes a distal race 152 and a proximal race 160. The distal race 152 is separate from and rotates relative to the proximal race 160 via a set of caged balls 154 (see FIG. 5) spaced arcuately therebetween. The caged balls 154 may attach to the proximal race 160. The thrust bearing 150 may be a variety of suitable thrust bearings configured to primarily absorb axial loads, or loads generally along the proximal/distal directions. The thrust bearing 150 transmits such axial loads during rotation of the digit 100, as further described herein, for example with respect to FIG. 5.

The distal race 152 defines an opening 156 therethrough. The proximal race 160 defines an opening 162 therethrough. The openings 156, 162 are configured to align with each other and to receive a proximal end of the shaft 120 therein. The opening 156 may be smooth and be located proximally and adjacent to the first attachment area 126 of the shaft 120. The opening 162 may be internally threaded and be located at the second attachment areca 128 of the shaft 120 to engage corresponding outer threads of the second attachment arca 128. When assembled, the distal race 152 contacts the outer race 146 of the radial bearing 140, and the proximal race 160 rotates with the shaft 120 relative to the distal race 152 via the caged balls 154, as further described herein, for example with respect to FIG. 5. The opening 162 of the proximal race 160 may threadingly engage the second attachment area 128, be bonded to the second attachment area 128, be mechanically attached in other suitable ways with the second attachment area 128, or combinations thereof.

In some embodiments, the proximal race 160 may be a cap or nut having an internal thread and that is configured to rotate relative to the distal race 152 via the caged balls located therebetween. For example, an internally threaded nut, a circular disc with an internal thread, or other suitable component may be used as the proximal race 160.

FIG. 4B is a partially exploded view of the actuator 300. The motor 112, gearbox 114, shaft 120, worm gear 120, and worm wheel 104 are assembled together. The radial bearing 140 and thrust bearing races 152, 160 are shown in exploded view and may be positioned onto the shaft 120 as described herein. FIG. 4C depicts the actuator 300 of FIG. 4B with the housing 106. The housing 106 covers the gearbox 114, worm gear 130, and shaft 120. The bearings 140, 150 attach to the shaft 120 and are also located inside the housing 106 when assembled, as more clearly shown in FIG. 5. As the digit 100 rotates, the housing 106, motor 112, gearbox 114, shaft 120, worm gear 120, and bearings 140, 140 rotate together about the firs axis 1 as the worm gear 120 travels along the teeth 105 of the worm wheel 104.

FIG. 5 is a side cross-section view of the prosthetic digit 100 including the actuator 300 assembled together. The motor 112 may be located within the distal portion 107 that is attached to the housing 106. The housing 106 and/or distal portion 107 may form a proximal segment of the digit 100. The distal portion 107 may rotate with the housing 106. The distal portion 107 may be removably attached to the housing 106 for ease of assembly and maintenance. The distal segment 108 may be rotatably attached to the distal portion 107 of the proximal segment 106. The distal portion 107 may have an opening therethrough, which may correspond to and continue the opening 109 of the housing 106.

As shown, a distal end of the inner race 142 of the radial bearing 140 contacts a proximal end of the worm gear 130 at a contact area 133. The contact area 133 may be a rounded, for example circular, surface area and extend about the longitudinal axis of the bearing 140. The worm gear 130 may transmit axial forces in the proximal direction to the housing 106 via the contact area 133. For example, as the digit 100 performs a closing rotation, such that the worm gear 130 travels counterclockwise relative to the worm wheel 104 as oriented in FIG. 5, the worm wheel 104 exerts axial forces on the worm gear 130 in the proximal direction. These proximal axial forces are then transmitted from the worm gear 130 to the inner race 142 via the contact area 133. The inner race 142 then transmits the forces via the balls of the bearing 140 to the outer race 146, which is in contact with the inner surface of the housing 106 and so transmits the forces to the housing 106. In this manner, axial forces in the proximal direction are transmitted to the housing 106. Further, these proximal axial forces are transmitted as the shaft 120 rotates because the inner race 142 may rotate with the shaft 120 and relative to the outer race 146. The outer race 146 may be rotationally stationary relative to the housing 106. The distal end of the outer race 146 may not contact the worm gear 130.

As further shown in FIG. 5, a distal-facing surface of the distal race 152 of the thrust bearing 150 contacts a proximal-facing surface of the proximal end of the outer race 146 of the radial bearing 140 at a contact area 151. The contact area 151 may be rounded, for example circular, and extend about the longitudinal axis of the bearing 150. The thrust bearing 150 may transmit axial forces in the distal direction to the housing 106 via the contact area 151. As the digit 100 performs an opening rotation, such that the worm gear 130 travels clockwise relative to the worm wheel 104 as oriented in FIG. 5, the worm wheel 104 exerts axial forces on the worm gear 130 in the distal direction. These distal axial forces are then transmitted from the worm gear 130 to the proximal race 160 of the thrust bearing 150 via the shaft 120. The proximal race 160 then transmits the forces via the balls of the bearing 150 to the distal race 152, which is in contact with the outer race 146 of the radial bearing 140 at the contact area 151, and so transmits the forces to the housing 106 via the flange 148 of the radial bearing 140.

In this manner, axial forces in the distal direction are transmitted to the housing 106. Further, these distal axial forces are transmitted as the shaft 120 rotates because the proximal race 160 rotates with the shaft 120 and relative to the distal race 152. The distal race 152 may be rotationally stationary relative to the outer race 146. The distal-facing surface or surfaces of the distal race 152 may be compressed against the proximal-facing surface or surfaces of the outer race 146. The elimination of axial play in the assembled components may cause such contact and compression. In some embodiments, the distal race 152 is bonded to the outer race 146 and/or housing 106, is mechanically attached in other suitable ways to the outer race 146 and/or housing 106, or combinations thereof. The distal race 152 may not contact the inner race 142 of the radial bearing 140. There may be a groove or recess in the distal-facing surface of the distal race 152, and/or the inner race 142 may extend proximally but stop short of contacting the distal-facing surface of the distal race 152.

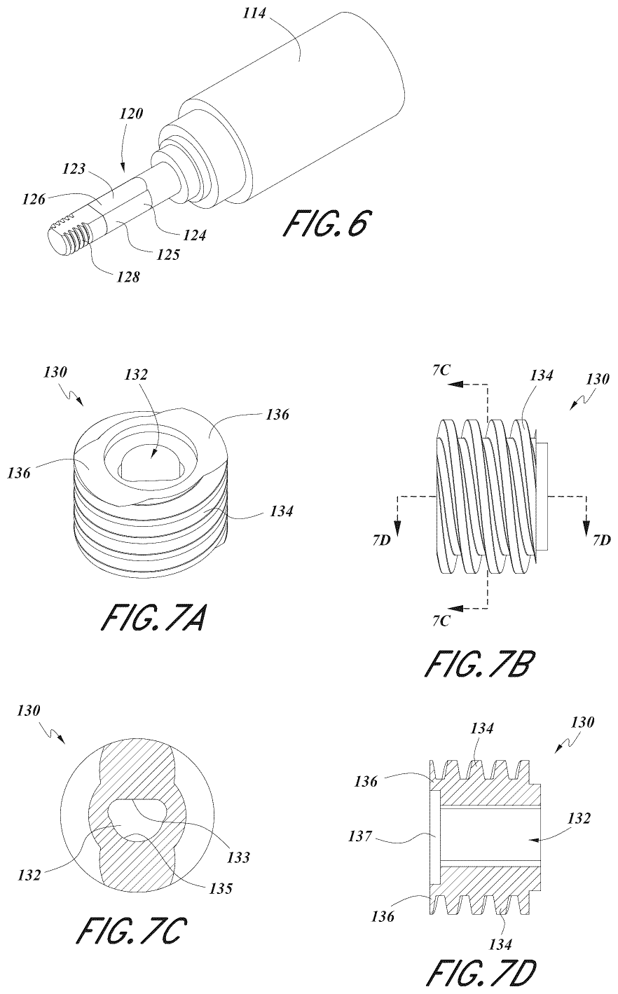

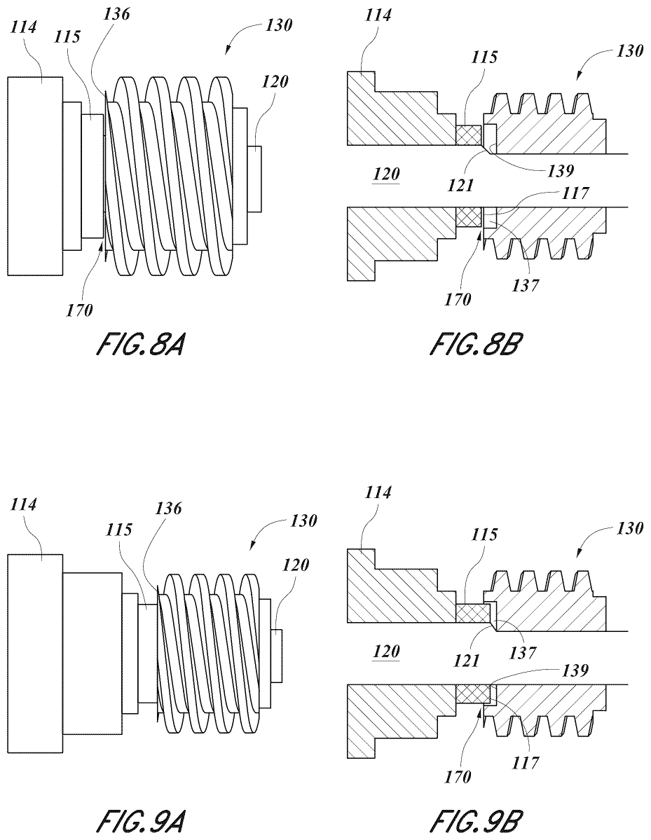

The actuator 300 may include a space 170. The space 170 may be a gap, opening, empty volume, or the like. The space 170 may be located on a distal side of the distal-facing surfaces of the distal end of the worm gear 130. The space 170 may be between the worm gear 130 and the gearbox 114. The worm gear 130 may thus be unsupported on a distal-facing end of the worm gear 130. The worm gear 130 may be axially fixed such that the space 170 remains while the shaft 120 is rotating and while stationary. The space 170 as measured axially, or as measured parallel to distal and proximal directions, between the proximal-most end of the gearbox 114 and the distal-most end of the worm gear 130 may be greater than or equal to 0.040 millimeter (mm). In some embodiments, the space 170 measured as described may be greater than or equal to 0.010 mm, greater than or equal to 0.020 mm, greater than or equal to 0.030 mm, greater than or equal to 0.035 mm, greater than or equal to 0.045 mm, greater than or equal to 0.050 mm, greater than or equal to 0.060 mm, greater than or equal to 0.080 mm, greater than or equal to 0.10 mm, or greater than or equal to 0.20 mm. In some embodiments, the space 170 may have other configurations, as described in further detail herein, for example with respect to FIGS. 8A-9B.

FIG. 6 is a partial perspective view of the actuator 300 showing the output shaft 120 assembled with the gear box 114. As shown, the shaft 120 may include the shaft portion 124 that extends outward from the gearbox 114. Further, the elongated shaft portion 124 may include a flat portion 123. The flat portion 123 may be a non-rounded contour. The flat portion 123 may be any contour that differs from the other surrounding outer contour of the shaft portion 124. The shaft portion 124 may include a rounded portion 125. Thus there may be the flat portion 123 surrounded by the rounded portion 125. The flat portion 123 and the rounded portion 125 may extend longitudinally along the length of the shaft portion 124. The flat portion 123 and/or the rounded portion 125 may extend to the tip or proximal end of the shaft portion 124, as shown. Thus, the second attachment area 128 may include the flat portion 123 and the rounded portion 125. The first attachment area 126 may also include the flat portion 123 and the rounded portion 125, as shown. The rounded portion 125 at the second attachment area 128 may be threaded, etc., as described herein.

The shaft portion 124 may have a “D” cross-sectional shape, with a flat side on an otherwise rounded cross-section. In some embodiments, there may be two or more flat portions 123, for example two flat portions 123 located opposite each other about an axis of the shaft portion 124. A variety of other non-circular cross-section shapes may be implemented that will provide for transmission of rotation forces to parts that are supported on the shaft portion 124. The shaft portion 124 may be polygonal, segmented, have multiple flat segments separated by multiple rounded or non-flat segments, other contours, or combinations thereof. The shaft portion 124 may be shaped to correspond and mechanically engage with the worm gear 130, the opening 144 of the inner race 142 of the radial bearing 140, and/or one or more of the openings 156, 162 of the thrust bearing 150. In some embodiments, the shaft portion 124 and corresponding openings of the various parts thereon may have a circular or other rounded cross-sectional shape. The shaft portion 124 and corresponding openings of the various parts thereon may be welded together. For example, the shaft portion 124 and the worm gear 130, and/or other parts, may be welded together and with corresponding circular cross-sectional shapes.

FIGS. 7A-7D are various views of the worm gear 130 of the actuator 300.

FIG. 7A is a perspective view, FIG. 7B is a side view, FIG. 7C is a cross-sectional view as taken along the line 7C-7C (shown in FIG. 7B), and FIG. 7D is a cross-sectional view as taken along the line 7D-7D (shown in FIG. 7B). The worm gear 130 includes the opening 132 having a non-circular cross-sectional shape. As shown in FIG. 7C, the opening 132 may include a flat portion 133 surrounded by one or more rounded portions 135. The opening 132 may have any of the cross-sectional shapes as described with respect to the shaft portion 124, for example with respect to FIG. 6. When assembled together, the flat portion 133 and rounded portion 135 of the worm gear 130 may correspond to and engage with, respectively, the flat portion 123 and rounded portion 125 of the shaft portion 124. Rotation of the shaft 120 will thus transmit rotational forces to the worm gear 130 via the non-rounded engagement of the respective surfaces. For example, the flat portions 123, 133 will engage each other to transmit rotation from the shaft portion 124 to the worm gear 130. Similar shaped openings may be included with the opening 144 of the inner race 142 of the radial bearing 140, and/or one or more of the openings 156, 162 of the thrust bearing 150. In some embodiments, the shaft portion 124 and the worm gear 130 may be welded together, such that rotation of the shaft will rotate the worm gear via the welded connection. The shaft portion 124 and the worm gear 130 may have circular or other rounded cross-sectional shapes and be welded together, as described. Such welding may also be included in non-circular or non-rounded cross-sectional shapes, such as the “D” cross-section or others described herein,

As shown in FIG. 7D, the worm gear 130 may include a recess 137 at an end thereof, for example at the distal end as assembled. The recess 137 may have an inner width that is greater than an inner width of the opening 132. The recess 137 may define a space on the end of the worm gear 130 to ensure that the distal end of the worm gear 130 does not contact the proximal end of the gearbox 114 (or proximal end of the motor 112 in embodiments not having the gearbox 114). The recess 137 may thus help define the space 170 located on the distal end of the worm gear 130 when assembled, as described herein, for example with respect to FIG. 5.

The recess 137 may be defined between one or more partial threads 136 at the end of the worm gear 130 that extend outwardly and radially away from an axis of the worm gear 130. The continuation of the spiral threads 134 become flat against a plane orthogonal to the axis of rotation. The partial threads 136 may be on the proximal and/or distal end of the worm gear 130. The recess 137 may be located on the proximal and/or distal end of the worm gear 130. The recess may allow for glue or weld overflow when assembling and/or manufacturing the shaft 120 and worm gear 130. In some embodiments, the recess 137 is located on the proximal end of the worm gear 130, and a spacer is used to orient the worm gear 130 against the distal end of the inner race 142 of the radial bearing 140. The spacer may be a thin, circular structure with an opening therethrough, e.g. similar to a washer.

FIGS. 8A-8B are side and cross-section views, respectively, of another embodiment of the space 170 between the worm gear 130 and gearbox 114 that may be implemented with the various actuators described herein. The actuator, such as the actuator 300, may include a bushing 115 located at a proximal end of the gearbox 114. The bushing 115 may have a proximal-facing surface 117. The bushing 115 may have an outer width, e.g. outer diameter.

The shaft 120 may include a ramp 121 located at a longitudinal station along the length of the shaft 120. The ramp 121 may be on the shaft portion 124. The ramp 121 may be a projection extending radially outwardly from the shaft portion 124. The ramp 121 may be located at a proximal end of the bushing 115. The ramp 121 may be a transition zone of the shaft 120 where the shaft 120 changes from a circular cross-section to a non-circular cross-section. In some embodiments, the shaft portion 124 may have a circular cross-section on both sides of the ramp 121.

The worm gear 130 may have the recess 137 with a floor 139, which may be a distal-facing surface as oriented in the figure. The floor 139 partially forms the recess 137, such as a depth thereof. The floor 139 may be axially separated from the proximal-most end of the ramp 121. The floor 139 may not contact the ramp 121. The floor 139 may located distally of the floor 139.

The recess 137 may have a width, e.g. diameter. The width may extend between opposing inner walls of the recess 137, e.g. between opposing inner walls of the partial threads 136. The proximal-most end of the bushing, such as the surface 117, may be separated from a distal-most surface of the worm gear 130, such as the partial thread 136. The space 170 may exist axially between the worm gear 130 and the bushing 115, for example between the distal-most surface of the worm gear and the proximal-most surface of the bushing 115. In embodiments where there is no bushing 115, similar arrangements may be implemented between the gearbox 114 and the worm gear 130. The space 170 may have any of the sizes described herein, for example with respect to FIG. 5.

FIGS. 9A-9B are side and cross-section views, respectively, of another embodiment of the space 170 between the worm gear 130 and gearbox 114 that may be implemented with the various actuators described herein. The embodiment of the space 170 shown in FIGS. 9A-9B may have similar features as the embodiment of the space 170 shown in FIGS. 8A-8B, except as otherwise described. The embodiments shown in FIGS. 8A-9B may include any of the features and functions of the various parts as described herein, for instance for the worm gear 130, shaft 120, etc., for example as described with respect to any of FIGS. 3A-7D. The worm gear 130 may therefore have a circular or non-circular opening, the shaft 120 may have a circular and/or non-circular cross-sectional shape, etc.

As shown in FIGS. 9A-9B, the distal-most end of the worm gear 130 may be located distally of the proximal-most end of the bushing 115, such as the bushing surface 117. Thus, when assembled, an axial gap between the worm gear 130 and the bushing 115 may not be visible. However, there may still be an axial gap between the bushing surface 117 and the floor 139. The space 170 may further include a gap between radial-facing surfaces of the worm gear 130 and the bushing 115. The outer width of the bushing 115 may be smaller than the inner width of the recess 137. Thus the space 170 may be between an outer surface of the bushing 115 and an inner wall or surface of the recess 137. The space 170 may therefore include axial and radial gaps between the opposing parts.

The shaft ramp 121 may contact the floor 139 of the worm gear 130. The floor 139 may abut a proximal end of the ramp 121. The ramp 121 may be a limiting structural feature, such as stop, for axially locating the worm gear 130 on the shaft 120. The ramp 121 may limit travel of the worm gear 130 in the distal direction. The worm gear 130 may bottom out on the ramp 121 and/or other structural features of the shaft 120. The worm gear 130 may bottom out and contact the ramp 121 and/or other structural features of the shaft 120 and also be attached to the shaft 120 in one or more of any of the other attachment methods described herein, such as welding, bonding, etc. The ramp 121 or portions thereof may be located proximally of the distal-most surface of the worm gear 130 when assembled. In embodiments where there is no bushing 115, similar arrangements may be implemented between a proximal end of the gearbox 114 and the worm gear 130. The space 170 may have any of the dimensions described herein, for example with respect to FIG. 5, albeit in a radial direction.

In some embodiments, the worm gear 130 may radially contact the bushing 115 but still have an axial space 170 therebetween. For example, the respective radially opposing surfaces of the outer surface of the bushing 115 and the inner surfaces of the worm gear 130, such as the radially inward facing surfaces of the recess 137, may contact each other when assembled. These surfaces may form an interference, friction and/or other type fit between them when assembled. There may still be a space 170 between axially opposing surfaces of the bushing 115 and the worm gear 130, such as between the proximal-facing surface of the bushing 115 and the distal facing surface of the floor 139 of the worm gear 130. Thus the worm gear 130 may be radially supported but axially unguided by the bushing 115.

FIGS. 10A-10G are various views of another embodiment of a prosthetic digit 400 having an actuator 500 with an output shaft 420 having a unibody worm gear 430. FIG. 10A is a bottom perspective view, FIG. 10B is a top perspective view, FIG. 10C is a side view, FIG. 10D is a side cross-section view, FIG. 10E is a side view of the digit 400 with some components shown transparently for clarity, and FIGS. 10F and 10G are respectively a side view and a bottom perspective view of the digit 400 showing a tendon and with certain components hidden for clarity. The digit 400 and components thereof may have the same or similar features and/or functions as the digit 100 and respective components thereof, and vice versa, except as otherwise described herein.

The digit 400 includes a base 402, a worm wheel 404, a housing 406, a distal portion 107, a distal segment 108, a first rotational axis, a second rotational axis, and an actuator 500, which may have the same or similar features and/or functions as, respectively, the base 102, the worm wheel 104, the housing 106, the distal portion 107, the distal segment 108, the first rotational axis, the second rotational axis, and the actuator 300 of the digit 100.

The digit 400 further includes a fairing 413. The fairing 413 covers the proximal end of the digit 400. The fairing 413 may be attached to the digit 400 about the axis 1. The fairing 413 may attach at two opposite sides of the digit 400 at the axis 1. The fairing 413 may extend along opposite sides of the digit 400 and around the digit 400 from one attachment point to the other. The fairing 413 may be spaced apart from the proximal end of the digit 400 such that the digit 400 can rotate underneath the fairing 413. The fairing 413 provides structural protection of the rotating digit 400, for example the rotating proximal end of the digit 400. The fairing 413 may be stationary. In some embodiments, the fairing 413 may rotate, for example about the axis 1. The fairing 413 may be removable and re-attachable, for example with a friction fit about the digit 400 or by other suitable mechanical attachment means. The fairing 413 may be removed to access the proximal end of the digit 400 for instance to adjust a proximal end 428 of the output shaft 420, as further described herein, for example with respect to FIG. 12.

As shown in FIG. 10D, the actuator 500 includes a worm gear 430 and a worm wheel 404, which may have the same or similar features and/or functions as, respectively, the worm gear 130 and the worm wheel 104. The worm gear 130 is unibody with the output shaft 420, as further described. The threaded worm gear 430 is rotated and in response moves along the outer threaded, rounded contour of the worm wheel 404, as described herein with respect to the gear 130 and wheel 104.

As further shown in FIG. 10D, the digit 400 includes an extension spring 401. The spring 401 extends from the distal portion 407 to the distal segment 408. A proximal end of the spring 401 may attach to the distal segment 408 and a distal end of the spring 401 may attach to the distal segment 408. The spring 401 may attach to a first rod 401A of the distal segment 408 and a second rod 401B of the distal portion 407. The spring 401 provides a biasing rotational force on the distal segment 408 of the digit 400 that rotationally biases the distal segment 408 to straighten out. The spring 401 may thus “pull” on the distal segment 408 toward the distal portion 407.

FIGS. 10D and 10F-10G show the digit 400 including a tendon 405. For clarity, certain structures of the digit 400 are hidden in FIGS. 10D and 10F-10G, including the housing of the distal portion 407, the proximal housing 406, and the fairing 413.

The tendon 405 is a tether or wire, which may be inelastic or substantially inclastic. In some embodiments, the tendon 405 may be clastic. The tendon 405 extends from or near the worm wheel 404, along the housing 406, and to a distal end of the distal portion 407. The tendon 403 may further extend to the distal segment 408. The tendon 405 may effectively shorten or lengthen the distance between the segments as the digit 400 rotates to cause the distal segment 408 to rotate relative to the proximal segment of the digit 400 having the housing 406. As the digit 400 rotates to open (or clockwise as oriented in FIGS. 10D and 10E), the tendon 405 may effectively lengthen and allow the distal segment 408 to straighten out via the extension spring 401. Conversely, as the digit 400 rotates to close (or counterclockwise as oriented in FIGS. 10D and 10E), the tendon 405 may effectively shorten pulling on the distal segment 408 to create a torque of the distal segment 408 about its pivot point with the proximal segment, thereby causing the distal segment 408 to also rotate counterclockwise relative to the proximal segment and toward the base 402.

As shown in FIGS. 10F and 10G, the tendon 405 extends from the worm wheel 404 along the length of the digit 400 to the distal segment 408. The tendon 405 has a proximal end 417 that attaches to the worm wheel 404. A set screw or other mechanical mechanism may secure the proximal end 417 to the worm wheel 404. The tendon 405 has a distal end 415 that attaches to or wraps around a portion of the distal segment 408. The tendon 405 may be continuous and include a first segment 405A and a second segment 405B extending from the worm wheel 404 to the distal segment 408, as shown in FIG. 10G. The tendon 405 may be secured at the proximal end 417 and extend along the first and second segments 405A, 405B to the distal end 415. The first segment 405A and the second segment 405B may extend from the worm wheel 404, distally underneath the proximal segment of the digit 400, along a pulley or rod, and to the distal segment 408. The distal end 415 may wrap around a rod or other attachment in the distal segment 408. The tendon 405 may extend a distance away from the axis 2 to create a torque about the axis 2 when rotating closed. The tendon 405 may be an inelastic or substantially inelastic member.

The tendon 405 and the extension spring 401 operate to cause the proximal and distal segments of the digit 400 to rotate open and closed as the worm gear 430 travels along the worm wheel 404. The extension spring 401 biases the digit 400 to open. The tendon 405 pulls on the digit to close. The digit 400 may be rotated open or closed by the actuator 500. In some embodiments, the digit 400 may be rotated open and closed by external forces, such as by an object exerting an external force on the digit 400.

When the actuator 500 causes a closing rotation, the worm gear 430 travels along the worm wheel in the clockwise direction as oriented in FIG. 10F to rotate the gearbox housing 414 clockwise about the Axis 1. The fixed proximal end 417 of the tendon 405 effectively shortens the length of the tendon 405, pulling on the distal end 415 of the tendon 405 to cause the distal segment 408 of the digit 400 to rotate clockwise in a closing direction about the Axis 2. As the digit 400 rotates to close, the extension spring 401 extends and stores a potential compressive restoring force to bias the digit 400 toward the open position. Similar action of the spring 401 and tendon 405 may operate when the digit 400 is rotated closed by an external force.

When the actuator 500 causes an opening rotation, the worm gear 430 travels along the worm wheel in the counterclockwise direction as oriented in FIG. 10F to rotate the gearbox housing 414 counterclockwise about the Axis 1. The fixed proximal end 417 of the tendon 405 effectively lengthens the length of the tendon 405, allowing the stored potential force of the spring 401 to cause the distal segment 408 of the digit 400 to rotate counterclockwise in an opening direction about the Axis 2. As the digit 400 rotates to open, the extension spring 401 contracts and releases the potential compressive restoring force. The open position may include the distal segment 408 forming an extended position that is parallel or near parallel to the housing 406 of the proximal segment of the digit 400. Similar action of the spring 401 and tendon 405 may operate when the digit 400 is rotated open by an external force.

As shown in FIG. 10E, the digit 400 includes an electrical support 403. The support 403 extends from the worm wheel 404, along the housing 406, and to a distal end of a motor 412. A pair of electrical leads 495 extend from respective proximal connections 496 to distal connections 497. The proximal connections 496 are electrically connected with conductive surfaces 498 of the worm wheel 404. The distal connections 497 are electrically connected with conductive surfaces of the motor 412. Power is supplied from a battery in the prosthetic hand to which the digit 400 is connected to the motor 412 via the electrical leads 495 as described. The proximal connections 496 may move over the conductive surfaces 498 as the digit 400 rotates to continuously provide power to the motor 412 during digit 400 rotation.

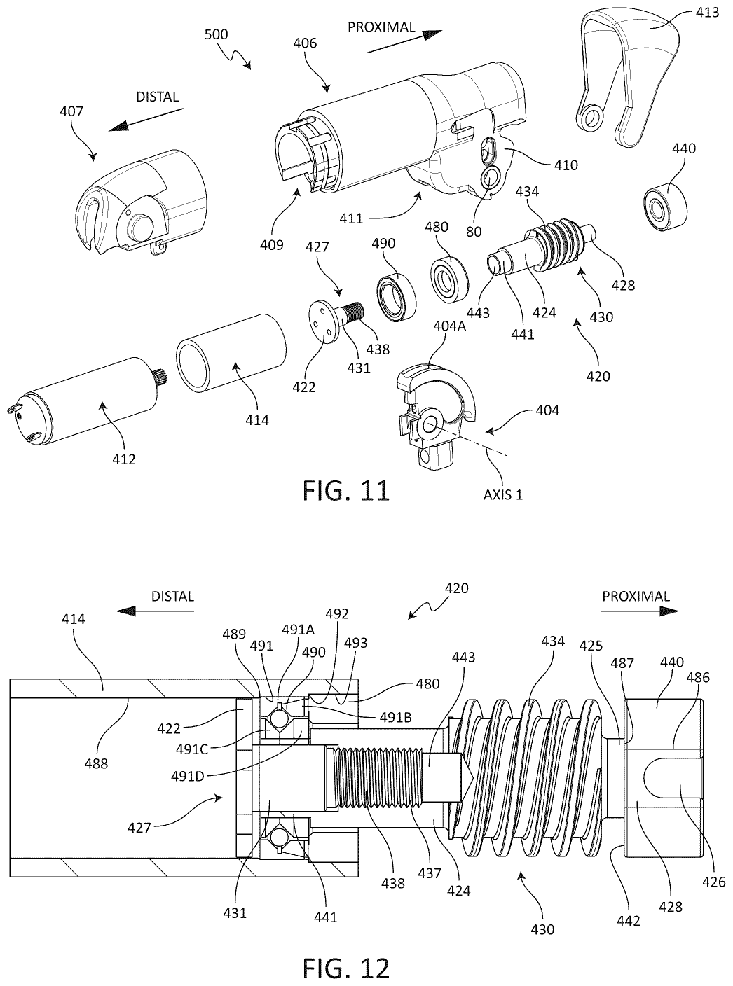

FIG. 11 is a partial exploded view of the actuator 500. FIG. 12 is a side cross-section view of the actuator 500 showing only some components for clarity, including an output shaft 420. The actuator 500 includes the motor 412 and a gearbox 414, which may have the same or similar features, respectively, as the motor 112 and the gearbox 114 of the digit 100. The actuator 500 may cause rotation of a prosthetic digit as described herein for other digits. Thus, the worm gear 430 may rotate about its own longitudinal axis to travel along teeth 404A of the worm wheel 404 and rotate about the axis 1, to thereby cause the proximal segment of the prosthetic digit, e.g. the housing 406, the motor 412, and/or the gearbox 414, to rotate about the axis 1 as well. The housing 406 and distal portion 407 define an opening 409 therethrough in which the motor 412, gearbox 414, and shaft 420 are located. The housing 406 includes a clevis 410 defining a space 411 therein and the opening 80, which may have the same or similar features and/or functions as, respectively, the clevis 410, space 411 and the opening 80 of the digit 100.

The digit 400 further includes an output carrier 427 having a larger diameter head 422 with a smaller diameter shaft 431 extending from the head 422. The carrier 422 may be part of a gearbox or set of gears within the gearbox 414. The gearbox 414 via the carrier 422 and other gears therein may transmit rotation from the motor 412 to the output shaft 420. The proximal end of the carrier 422 has an external thread 438.

The output shaft 420 includes a rounded distal portion 424 and a rounded proximal portion 425. In between the distal and proximal portions 424, 425 is the worm gear 430 having threads 434. The threads 434 may have the same or similar features and/or functions as the threads 134 of the digit 100. The worm gear 430 and the shaft portions 424, 425 are unibody. Thus the worm gear 430 and the shaft portions 424, 425 are a single, monolithic piece. The worm gear 430 and the shaft portions 424, 425 may be machined from the same piece of material, or they may be welded together, or they may be 3D-printed as a single piece. Other suitable fabrication methods may be employed to create the unibody shaft 420. The worm gear 430 may therefore not move axially relative to the shaft portions 424, 425. There may not be any guidances or other structures contacting either axial side of the worm gear 430.

The shaft 420 further includes a distal end 441. The distal end 441 extends distally from the distal shaft portion 424. The distal end 441 has a stepped-down (smaller) outer diameter than the distal shaft portion 424. The distal end 441 and the distal portion 424 may have a similar inner diameter to receive the carrier shaft 431 therein. The distal end 441 may also be unibody with the other features of the shaft 420. The distal end 441 and the portions 424, 425 may have circular cross-sections.

The distal end 441 may define a distal opening 443 therethrough. The opening 443 may extend into the distal portion 424. The opening 443 may have internal threads 437 along a portion thereof. The internal threads 437 may be located proximally of the distal end 441. The opening 443 may extend distally of the threads 437, for example to a location within a distal portion of the external threads 434 of the shaft 420. This is one example configuration, and the internal threads 437 and the extent of the opening 443 may be located axially along the shaft 420 in other locations. In some embodiments, the opening 443 may extend completely through the shaft 420. As shown, the shaft 420 includes a proximal opening 426 that protrudes slightly into the proximal end of the shaft 420, and the shaft 420 is solid between the two openings 443, 426.

The opening 443 of the shaft 420 may receive the shaft 431 of the carrier 427 therein. The external threads 438 of the carrier shaft 431 may mate with corresponding internal threads 437 of the shaft 420. The carrier 427 and the shaft 420 may be rotated relative to each other to cause the threads 437, 438 to engage and thereby engage the shaft 420 with the carrier 427.

The carrier head 422 and part of the carrier shaft 431 are located within the housing of the gearbox 414. The gearbox 414 includes a first diameter section 488 and a second relatively larger diameter section 491 with a radial step 489 therebetween. Similarly, the gearbox 414 includes the second diameter section 491 and a third relatively larger diameter section 493 with a radial step 492 therebetween. Thus the third diameter section 493 is wider than the second diameter section 491 which is wider than the first diameter section 488.

The actuator 500 further includes a distal bearing 490. The distal bearing 490 is a 4-point contact bearing that can take up both radial and axial loads. In some embodiments, other types of bearings or combinations of different types of bearings may be used for the distal bearing 490. The distal bearing 490 is located within the housing of the gearbox 414 within the second diameter section 491 with a distal end of the bearing 490 resting on the step 489.

The distal bearing 490 may have one or more outer races and one or more inner races. As shown, first and second outer races 491A, 491B of the bearing 490 contact the inner sidewall of the gearbox 414 housing and first and second inner races 491C, 491D of the bearing 490 contact the outer surfaces of the distal end 441 of the shaft 420. The inner races 491C, 491D rotate with the shaft 420 relative to the outer races 491A, 491B. The outer races 491A, 491B may be stationary relative to the gearbox housing as the inner races 491C, 491D rotate. The first outer race 491A may be located distally of the second outer race 491B. The first outer race 491A may contact the step 489, which may prevent axial travel of the bearing 490 in the distal direction. The second outer race 491B may contact and be compressed by the preload ring 480. The second outer race 491B may be rotationally stationary relative to the preload ring 480 and/or the housing 414.

The second inner race 491D may be located proximally of the first inner race 491C. The second inner race 491D may contact the step 432 of the shaft 420. The step 432 may be a radially extending outer surface connecting the relatively smaller outer diameter distal end 441 and the relatively larger outer diameter distal portion 424 of the output shaft 420.

The proximal end of the bearing 490 may be located slightly distally of the radial step 492 such that a bearing preload ring 480 contacts the proximal end of the bearing 490 to axially secure the bearing 490 within the gearbox 414. The ring 480 may contact one or more outer races of the bearing 490 such that the one or more inner races of the bearing 490 can rotate free of interference from the ring 480. The ring 480 is secured and constrained by the housing of the gearbox 414. A proximal end of the ring 480 may align with the proximal end of the gearbox 414.

The actuator 500 includes a proximal bearing 440. The bearing 440 may be a radial bearing configured to take up radial loads at the proximal end of the shaft 420. The bearing 440 is located at the proximal end 428 of the shaft 420. The bearing 440 is located on a relatively smaller diameter section 486 of the proximal end 428 with respect to the proximal portion 425 of the shaft 420. A radial step 487 is located between the proximal portion 425 and the section 486. The bearing 440 is axially located next to the step 487. One or more shims 442 may be used between the bearing 440 and the step 487 to finely axially align the bearing 440.

Various modifications to the implementations described in this disclosure can be readily apparent to those skilled in the art, and the generic principles defined herein can be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system components in the implementations described above should not be understood as requiring such separation in all implementations, and it should be understood that the described program components and systems can generally be integrated together in a single software product or packaged into multiple software products. Additionally, other implementations are within the scope of the following claims. In some cases, the actions recited in the claims can be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. (canceled)

2. A method of actuating a prosthetic digit, the method comprising:
rotating a shaft and worm gear about a first axis defined by the shaft, wherein the worm gear does not move axially relative to the shaft;
moving the worm gear along an arcuate perimeter of a worm wheel in response to rotation of the worm gear; and
rotating a gearbox housing about a second axis that is angled with respect to the first axis, wherein the rotating of the gearbox housing is in response to moving the worm gear along the arcuate perimeter of the worm wheel, and wherein the gearbox housing defines a gap between a proximal facing side of a proximal end of the gearbox housing and a distal facing side of a distal end of the worm gear.

3. The method of claim 2, further comprising rotating a bearing within the gearbox housing, wherein the bearing supports the shaft.

4. The method of claim 3, further comprising axially compressing the bearing by application of a compressive force on a proximal side of the bearing.

5. The method of claim 3, further comprising axially compressing the bearing by application of a compressive force on a distal side of the bearing.

6. The method of claim 2, wherein the worm gear is unibody with the shaft.

7. The method of claim 2, wherein the shaft comprises an output shaft, and wherein rotating the output shaft is via rotation of a carrier shaft.

8. The method of claim 2, further comprising rotating the shaft via a motor and a gearbox.

9. The method of claim 2, further comprising causing the prosthetic digit to perform an opening rotation.

10. The method of claim 2, further comprising maintaining the gap during rotation of the shaft.

11. A method of actuating a prosthetic digit, the method comprising:
rotating a longitudinal shaft about a first axis defined by the shaft to cause a worm gear to rotate about the first axis, wherein the worm gear remains axially stationary along the shaft as the worm gear rotates;
translating the worm gear along a rounded worm wheel in response to rotation of the worm gear to cause a proximal segment of the prosthetic digit to rotate about a second axis that is different from the first axis;
rotating a bearing that is supporting the shaft; and
axially compressing the bearing.

12. The method of claim 11, wherein the bearing supports the shaft within a gearbox housing.

13. The method of claim 12, wherein a distal side of the bearing is axially compressed by the gearbox housing.

14. The method of claim 12, wherein a proximal side of the bearing is axially compressed by a preload ring within the gearbox housing.

15. The method of claim 11, wherein the second axis is perpendicular to the first axis.

16. The method of claim 12, wherein the gearbox housing defines a gap between a proximal facing side of a proximal end of the gearbox housing and a distal facing side of a distal end of the worm gear.

17. The method of claim 11, wherein the worm gear is unibody with the shaft.

18. A method of actuating a prosthetic digit, the method comprising:
rotating a shaft and worm gear about a first axis defined by the shaft;
engaging threads of the worm gear with teeth of a worm wheel to cause the worm gear to travel along the worm wheel in response to rotation of the worm gear;
rotating a gearbox housing about a second axis that is angled with respect to the first axis and in response to the worm gear traveling along the worm wheel, wherein the gearbox housing defines a gap between a proximal end of the gearbox housing and a distal end of the worm gear; and
axially compressing a bearing that supports the shaft inside the gearbox housing.

19. The method of claim 18, further comprising the worm gear remaining axially stationary along the shaft as the gearbox housing rotates about the second axis.

20. The method of claim 18, further comprising axially compressing the bearing with the gearbox housing and a preload ring.

21. The method of claim 18, further comprising causing the prosthetic digit to perform an opening rotation.
